(19) United States
(12) Patent Application Publication
(10) Pub. No.: US 2007/0065382 A1
(43) Pub. Date: Mar. 22, 2007

(54) Cosmetic composition comprising at least one active agent and particles containing at least one colored inorganic pigment in a matrix, and skincare use thereof

(76) Inventor: Guillaume Cassin, Villebon Sur Yvette (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/520,760

(22) Filed: Sep. 14, 2006

(30) Foreign Application Priority Data
Sep. 14, 2005 (FR) 05 09370

(51) Int. Cl. A61K 8/19; A61K 8/29

(52) U.S. Cl. 424/63; 424/59

(57) ABSTRACT

Disclosed herein is a cosmetic composition for treating and/or caring for the skin, which comprises, in a physiologically acceptable medium, at least one cosmetic active agent and at least one colored particle comprising at least one inorganic pigment arranged inside a matrix, wherein the composition has an opacity of less than or equal to 35%. Also disclosed herein is a cosmetic treatment process comprising applying this composition to keratin materials. Further disclosed herein is a method for lightening and/or unifying the complexion of the skin and/or for correcting the appearance of skin marks and dyschromias comprising applying a composition of the present disclosure to the skin.

This application claims benefit of priority under 35 U.S.C. § 119 to French Patent Application No. FR 05 09370, filed Sep. 14, 2005, the contents of which are incorporated herein by reference.

Disclosed herein are novel cosmetic compositions for treating and/or caring for the skin: for example, compositions for giving the skin a uniform color. The compositions according to the present disclosure are non-covering and comprise at least one active principle and colored particles comprising at least one inorganic pigment arranged inside a matrix.

It is common for people with colored skin, with pigmentation marks, couperose, and/or rings under the eyes, to wish to correct these cutaneous dyschromias, and use for this purpose cosmetic and/or dermatological compositions for lightening and/or unifying the complexion. To this end, it is known practice to use cosmetic compositions comprising bleaching agents. However, it is often necessary to use these agents for a long time and in large amounts in order to observe a bleaching effect on the skin, since no immediate effect is observed when these compositions are applied.

For the purpose of immediate lightening and unifying of the complexion, it is known practice to use cosmetic compositions comprising fluorescent compounds, for example, optical brighteners. However, these compounds afford an immediate lightening effect only under optimum lighting conditions (natural light of strong intensity).

For the purpose of immediate lightening and unifying of the complexion, it is also known practice to use covering products, which, although hiding the skin imperfections, have the major drawback of masking the natural appearance of the skin (mask sensation).

Finally, it is known practice to use products containing interference pigments, which, although being able to hide skin imperfections, have the major drawback of giving the skin an unnatural shiny appearance.

There is thus still a need for non-covering cosmetic compositions that have a lightening or unifying effect on the complexion irrespective of the nature of the lighting, and which do not give the skin a shiny and/or unnatural appearance.

Disclosed herein is a cosmetic composition for treating and/or caring for the skin that avoids at least one of the drawbacks of the compositions of the prior art, for example, a composition that is non-covering and that allows dyschromias to be corrected, i.e., that allows the skin complexion to be lightened and/or unified.

The present inventor has discovered, advantageously and unexpectedly, that compositions comprising at least one cosmetic active agent and colored particles comprising at least one inorganic pigment arranged inside a matrix (referred to herein as “first colored particles”) may solve at least one of the drawbacks of the prior art compositions.

The colored particles used in the compositions according to the present disclosure have been used in the prior art, for example, U.S. Patent Application Publication No. 2005-0129638, in skin makeup compositions. However, this published application does not disclose skincare compositions, such as the compositions in accordance with the present disclosure, which, although being non-covering, can give a uniform skin complexion.

The compositions according to the present disclosure may make it possible to correct dyschromias and to obtain an immediate shadow-concealing effect or an immediate complexion lightening effect.

The compositions according to the present disclosure may also have the advantage of being non-covering and not giving the skin an unnatural shiny appearance.

According to a first embodiment, the cosmetic composition for treating and/or caring for the skin may comprise, in a physiologically acceptable medium, (i) at least one first colored particle comprising at least one inorganic pigment arranged inside a matrix, and (ii) at least one bleaching cosmetic active agent for the skin.

The compositions in accordance with this embodiment may make it possible to obtain uniform coloration of the complexion not only immediately after application but also over a longer period.

According to a second embodiment, the cosmetic composition for treating and/or caring for the skin may comprise, in a physiologically acceptable medium,
(i) an aqueous continuous phase,
(ii) at least one cosmetic active agent for the skin, and
(iii) at least one first colored particle comprising at least one inorganic pigment arranged inside a matrix,
   wherein the composition has an opacity of less than or equal to 35%.

It is often difficult to obtain good dispersion of inorganic pigments in an aqueous phase, and consequently it is difficult to obtain homogeneous compositions, for example, when the pigment concentration in these compositions is low. The compositions in accordance with this second embodiment may comprise an aqueous continuous phase and inorganic pigments encapsulated in a matrix. These compositions may have the advantage of being homogeneous and allowing a uniform coloration of the complexion to be obtained.

According to a third embodiment of the present disclosure, the cosmetic composition for treating and/or caring for the skin may comprise, in a physiologically acceptable medium,
(i) at least one cosmetic active agent for the skin, and
(ii) at least one first colored particle comprising at least two inorganic pigments of different colors arranged inside a matrix,
   wherein the composition has an opacity of less than or equal to 35%.

Generally, when it is desired to obtain a composition comprising a mixture of inorganic pigments, it is necessary to prepare a pigmentary paste, which is introduced into a base composition, but it is often difficult to obtain a homogeneous pigmentary paste, especially when it is desired to obtain a composition with a low concentration of pigments. The use of colored particles comprising at least two inorganic pigments arranged inside a matrix may make it possible to dispense with the step of preparing a pigmentary paste, and the compositions in accordance with this third embodiment may have the advantage of being homogeneous and allowing a uniform coloration of the complexion to be obtained.

The cosmetic active agent used in the compositions in accordance with the second and third embodiments may be a bleaching cosmetic active agent, and these compositions may allow a uniform coloration of the complexion to be obtained not only immediately after application but also over a longer period.

The compositions in accordance with the first and third embodiments may have an aqueous continuous phase.

Other characteristics, aspects, subjects, and advantages of the present disclosure will be understood more clearly upon reading the description and the examples that follow.

The compositions according to the present disclosure are compositions for treating and/or caring for the skin and are intended to be used to lighten and/or unify the complexion. The compositions may be non-covering, i.e., allow the grain of the skin to show through while at the same time masking its imperfections, and more generally may allow skin dyschromias to be corrected.

The compositions in accordance with the present disclosure may have an opacity of less than 35%, for example, ranging from 5% to 35%, or from 10% to 30%.

The compositions according to the present disclosure may have a color difference (ΔE) after application to a contrast card (Erichsen 24/5) of greater than 30.

In addition, the content of free inorganic pigments in the compositions in accordance with the invention may be very low. In at least one embodiment, the composition may contain less than 0.2% by weight of free inorganic pigments relative to the total weight of the cosmetic composition.

Although they are non-covering, the compositions according to the present disclosure may make it possible to give a uniform skin complexion and to satisfactorily correct skin dyschromias.

The compositions according to the present disclosure may comprise first colored particles comprising at least one colored inorganic pigment arranged inside a matrix.

Thus, as used herein, the term “first colored particles” according to the present disclosure does not include particles whose surface is covered with at least one layer of pigments. The “first colored particles” also differ from particles, for example, lamellar particles, with a multilayer interference structure.

As used herein, the term “first colored particles” means particles having a color other than white.

In the first colored particles according to the invention, the inorganic pigments are uniformly dispersed inside the matrix constituting the particles, for example, in the form of isolated particles.

The inorganic pigments may be dyestuffs that are insoluble in the matrix in which they are present.

In at least one embodiment, the compositions in accordance with the present disclosure may comprise first colored particles comprising at least two, for example, two or three, inorganic pigments of different colors arranged inside a matrix.

The compositions in accordance with the present disclosure may have the advantage of having a uniform, precisely-defined color that is readily reproducible, even when the concentration of composite particles is low.

These compositions may ensure that the user will obtain a precise and uniform coloration of the support onto which they are applied, the support thus being uniformly colored in the precise shade that the user has chosen beforehand.

The processes for obtaining particles of this type are described, for example, in Japanese patent documents JP-B-2591946 (Sumitomo Chemicals), JP-B2861806 (NSG), JP-A-H08-239310 (NSG), JP-A-H06-47273 (Suzuki Oil and Fat) in the case where the material forming the particles is inorganic, and JP-A-2001-354776 (Shin Etsu Chemicals) in the case where the material forming the particles is organic.

As used herein, the term “colored inorganic pigments” means metal oxide pigments having a color which may be chosen from red, yellow, blue, green, violet, brown, and black. Non-limiting examples of colored inorganic pigments include iron oxide, ultramarine, Prussian blue, ferric blue, manganese violet, and chromium oxide. Other examples of colored inorganic pigments include composite or mixed pigments comprising a mixture of metal oxides, for instance, mixed iron and titanium oxides. These pigments may have a size ranging from 0.1 to 1.5 microns.

The composite particles used according to the present disclosure may also comprise a mixture of colored inorganic pigments or a mixture of colored inorganic pigments and of white inorganic pigments. In at least one embodiment, the composite particles used according to the present disclosure may comprise a mixture of three inorganic pigments: a red inorganic pigment, a yellow inorganic pigment, and a white inorganic pigment. According to another embodiment, the inorganic pigments in the matrix of the first colored particles may be present in an amount ranging from 3% to 60% by weight relative to the total weight of the first colored particles.

There is no limitation in terms of shape of the first colored particles used according to the present disclosure. They may have a shape chosen, for example, from spheres, flakes, solid cylinders, and needles. The first colored particles used according to the present disclosure may be porous or non-porous. In at least one embodiment, at least one of the constituents of the first particles used according to the present disclosure should have a refractive index of less than 1.8, such as less than 1.6. The first colored particles used according to the present disclosure may have a mean volume size ranging from 1 to 40 microns as measured with a laser granulometer, for instance, a Mastersizer 2000® from Malvern and BI90+® from Brookhaven Instrument Corporation.

The matrix of the first colored particles may be composed of any suitable material, for instance, polymethyl methacrylate, cellulose, and porous silica.

Examples of commercially available first colored particles comprising at least one colored inorganic pigment include, but are not limited to, polymethyl methacrylate particles, for example, those sold under the name GANZPEARL by the company Ganz Chemicals, cellulose particles, for example, those sold under the name CELLULOBEADS by the company Daito, porous silica particles, for example, those sold under the name PC BALL by the company Miyoshi, and porous silica particles, for example, those sold under the name GOD BALL PC-LS by the company Suzuki Oil and Fat.

The composition according to the present disclosure may comprise from 0.1% to 10%, for example, from 0.5% to 5% by weight of first colored particles relative to the total weight of the composition.

The (bleaching) cosmetic active agent according to the present disclosure may be chosen from (bleaching) cosmetic active agents for the skin.

Non-limiting examples of cosmetic active principles that may be used in the compositions according to the present disclosure include, but are not limited to, antioxidants, sunscreens, vitamins, moisturizing active agents, self-tanning compounds, anti-wrinkle active agents, hydrophilic or lipophilic active agents, anti-pollution agents, free-radical scavengers, dermo-relaxing active agents, calmatives, agents for stimulating the synthesis of dermal or epidermal macromolecules and/or for preventing their degradation, anti-glycation agents, anti-irritant agents, desquamating agents, NO-synthase inhibitors, agents for stimulating fibroblast or keratinocyte proliferation and/or keratinocyte differentiation, agents acting on the capillary circulation, agents acting on the energy metabolism of cells, cicatrizing agents, and mixtures thereof.

In at least one embodiment, the compositions according to the present disclosure may comprise an anti-aging cosmetic active agent chosen from compounds that increase collagen synthesis, compounds that inhibit collagen degradation, dermo-decontracting agents, protein glycation inhibitors, agents for increasing keratinocyte proliferation, agents for increasing keratinocyte differentiation, and mixtures thereof.

Examples of bleaching cosmetic active principles that may be used in the compositions according to the present disclosure include, but are not limited to, depigmenting agents, anti-pigmenting agents, pro-pigmenting agents, and mixtures thereof.

The depigmenting or anti-pigmenting agents that may be incorporated into the composition according to the present disclosure may comprise, for example, at least one of the following compounds: kojic acid; ellagic acid; arbutin and derivatives thereof such as those described in European Patent Application Nos. 0 895 779 and 0 524 109; hydroquinone; aminophenol derivatives such as those described in International Patent Application Publication Nos. WO 99/10318 and WO 99/32077, for example, N-cholesteryloxycarbonyl-para-aminophenol and N-ethyloxycarbonyl-para-aminophenol; iminophenol derivatives, for example, those described in International Patent Application Publication No. WO 99/22707; L-2-oxothiazolidine-4-carboxylic acid or procysteine, and salts and esters thereof; calcium D-pantethine sulfonate, and ascorbic acid and derivatives thereof, for instance, ascorbyl glucoside; and plant extracts, such as extracts of liquorice, mulberry, scullcap, and Bacopa monnieri.

Pro-pigmenting agents may include the extract of pimpernel (Sanguisorba officinalis) sold by the company Maruzen and extracts of chrysanthemum (Chrysanthemum morifolium).

Non-limiting examples of anti-aging cosmetic active principles that may be used in the compositions according to the present disclosure may include:

compounds that increase collagen synthesis (such as extracts of Centella asiatica; asiaticosides and derivatives; ascorbic acid or vitamin C and derivatives thereof; synthetic peptides such as iamin, biopeptide CL, and palmitoyloligopeptide sold by the company Sederma; peptides extracted from plants, such as the soybean hydrolysate sold by the company Coletica under the trade name Phytokine®; and plant hormones such as auxins and lignans) and/or elastin synthesis (such as the extract of Saccharomyces cerivisiae sold by the company LSN under the trade name Cytovitin®; and the extract of the alga Macrocystis pyrifera sold by the company Secma under the trade name Kelpadelie®) and/or glycosaminoglycan synthesis (such as the product of fermentation of milk with Lactobacillus vulgaris, sold by the company Brooks under the trade name Biomin yogourth®; the extract of the brown alga Padina pavonica sold by the company Alban Müller under the trade name HSP3®; and the extract of Saccharomyces cerevisiae available, for example, from the company Silab under the trade name Firmalift® or from the company LSN under the trade name Cytovitin®) and/or proteoglycan and/or fibronectin synthesis (such as the extract of the zooplankton Salina sold by the company Seporga under the trade name GP4G®; the yeast extract available, for example, from the company Alban Müller under the trade name Drieline®; and the palmitoyl pentapeptide sold by the company Sederma under the trade name Matrixil®) and/or laminin synthesis;

compounds for inhibiting collagen degradation (such as retinoids and derivatives, oligopeptides and lipopeptides, lipoamino acids, the malt extract sold by the company Coletica under the trade name Collalift®; extracts of blueberry and rosemary; lycopene; isoflavones, derivatives thereof and plant extracts containing them, for instance, soybean extracts sold, for example, by the company Ichimaru Pharcos under the trade name Flavosterone SB®, and the extracts of red clover, flax, kakkon, and sage) and/or elastin degradation (such as the peptide extract of Pisum sativum seeds sold by the company LSN under the trade name Parelastyl®; heparinoids; and pseudodipeptides such as {2-[acetyl(3-trifluoromethylphenyl)amino]-3-methylbutyrylamino}acetic acid);

dermo-decontracting agents, such as alverin and salts thereof, manganese salts, for example, manganese gluconate, magnesium salts, such as magnesium gluconate sulfate, the hexapeptide Argireline R sold by the company Lipotec, adenosine, and sapogenins and natural extracts, for instance, of Dioscorea opposita and of Dioscorea villosa (wild yam) containing them, and extracts of Boswellia serrata;

protein glycation inhibitors, such as plant extracts of the Ericacea family, for example, extracts of blueberry (Vaccinium angustifolium); ergothioneine and derivatives thereof; and hydroxystilbenes and derivatives thereof, such as resveratrol and 3,3′,5,5′-tetrahydroxystilbene;

agents for increasing keratinocyte proliferation (such as retinoids, including retinol and retinyl palmitate, adenosine, phloroglucinol, walnut cake extracts sold by the company Gattefosse, and the extracts of Solanum tuberosum sold by the company Sederma) and/or fibroblast proliferation (such as plant proteins and polypeptides, extracted, for example, from soybean, and plant hormones such as giberrellins and cytokinins);

agents for increasing keratinocyte differentiation, such as minerals, including calcium; peptide extracts of lupin, such as the product sold by the company Silab under the trade name Structurine®; sodium β-sitosteryl sulfate such as the product sold by the company Seporga under the trade name Phytocohesine®; and water-soluble extracts of corn such as the product sold by the company Solabia under the trade name Phytovityl®; peptide extracts of Voandzeia subterranea such as the product sold by the company Laboratoires Sérobiologiques under the trade name Filladyn LS 9397®; and lignans such as secoisolariciresinol;

and mixtures thereof.

In at least one embodiment, the at least one anti-aging cosmetic active principle may be chosen from ascorbic acid and/or derivatives thereof such as ascorbyl glucoside, and adenosine, and mixtures thereof.

The at least one bleaching active principle may be present in the composition according to the present disclosure in an amount ranging from 0.001% to 10%, for example, from 0.01% to 5% by weight relative to the total weight of the composition.

The compositions according to the present patent application may also comprise additional second colored particles in an amount of less than or equal to 0.2% by weight relative to the total weight of the cosmetic composition.

In at least one embodiment, the additional second colored particles may be chosen from organic pigments and inorganic pigments, and mixtures thereof.

In another embodiment, the inorganic pigments used as additional second colored particles are “free” inorganic pigments, i.e., they are not incorporated into a particle.

The inorganic pigments that may be used in the compositions according to the present disclosure may be chosen from red, yellow, blue, green, violet, brown, and black metal oxide pigments. The colored inorganic pigments may be chosen from iron oxide, ultramarine, Prussian blue, ferric blue, manganese violet, chromium oxide, and mixed iron and titanium oxides.

According to at least one embodiment, the compositions according to the present disclosure may further comprise matting fillers and/or nacres of small size.

As used herein, the term “matting filler” denotes a spherical or non-spherical, porous or non-porous particle with a refractive index of less than or equal to 2.2, for example, less than or equal to 2, less than or equal to 1.8, or ranging from 1.3 to 1.6. The “matting fillers” according to the present disclosure may have a volume size comparable to that of the nacres used. The size of the fillers may thus be less than 15 microns measured with a laser granulometer, for instance, a Mastersizer 2000® from Malvern or a BI90+ from Brookhaven Instrument Corporation.

In one embodiment of the present disclosure, the “matting fillers” may be spherical.

In another embodiment of the present disclosure, the “matting fillers” may be porous. In this case, the specific surface area of the particles—which may be related to the porosity—is greater than 10 m²/g, for example, greater than 50 m²/g.

The matting nature of the fillers according to the invention may be defined by means of a gonioreflectometer measurement. To do this, a composition containing 5% fillers is spread onto a contrast card (Prufkarte type 24/5-250 cm² sold by the company Erichsen) using a mechanical film spreader (wet thickness of 30 microns). The composition is then dried overnight at a temperature of 37° C., and the reflection is then measured using a gonioreflectometer. The result obtained is the ratio R between the specular reflection and the diffuse reflection. The value of R is proportionately smaller the greater the matting effect. The matting fillers according to the invention are those which, at a content of 5% in a cosmetic composition, give a value of R of less than 1, for example, less than 0.75.

These fillers may be chosen, for example, from:

porous silica microparticles, for instance the silica beads SB150 and SB700 from Miyoshi with a mean size of 5 microns; the series-H Sunspheres from Asahi Glass, for instance SUNSPHERES H33, H51, and H53 with respective sizes of 3, 5 and 5 microns;

polytetrafluoroethylene powders, for instance the PTFE CERIDUST 9205F from Clariant, with a mean size of 8 microns;

silicone resin powders, for instance the silicone resin TOSPEARL 145A from GE Silicone, with a mean size of 4.5 microns;

hollow hemispherical silicone particles such as those described in European Patent Application No. 1 530 961;

acrylic copolymer powders, such as those of polymethyl(meth)acrylate, for instance the PMMA JURYMER MBI particles from Nihon Junyoki, with a mean size of 8 microns, the hollow PMMA spheres sold under the name COVABEAD LH85 by the company Wackherr, and the vinylidene chloride/acrylonitrile/methylene methacrylate expanded microspheres sold under the name EXPANCEL;

wax powders, for instance the paraffin wax particles MICROEASE 114S from MicroPowders, with a mean size of 7 microns;

polyethylene powders, for example, those comprising at least one ethylene/acrylic acid copolymer, and those comprising ethylene/acrylic acid copolymers, for instance the FLOBEADS EA 209 particles from Sumitomo (mean size of 10 microns);

crosslinked elastomeric organopolysiloxane powders coated with silicone resin, such as silsesquioxane resin, as described, for example, in U.S. Pat. No. 5,538,793. Such elastomeric powders are sold under the names KSP-100, KSP-101, KSP-102, KSP-103, KSP-104, and KSP-105 by the company Shin-Etsu;

talc/titanium dioxide/alumina/silica composite powders, for instance those sold under the name COVERLEAF AR-80 by the company Catalyst & Chemicals;

polyamide (Nylon®) powders, for instance the Nylon 12 particles of the ORGASOL type from Atofina, with a mean size of 10 microns;

and mixtures thereof.

The at least one matting filler may be present in the composition in an amount, depending on the desired effect, ranging from 0.1% to 15%, for example, from 0.5% to 10% by weight relative to the total weight of the composition.

The composition according to the present disclosure may also contain nacres of small size.

As used herein, the term “nacres” denotes particles in the form of a multitude of fine platelets with a high refractive index, which each reflect and partially transmit the incident light, these particles also occasionally being known as “interference pigments.”

The color effects obtained are associated with the lamellar structure of these particles and are derived from the physical laws of the optics of thin layers (see for example: Pearl Lustre Pigments—Physical principles, properties, applications—R. Maisch, M. Weigand. Verlag Moderne Industrie). The nacres used according to the present disclosure may be pigments of multilayer type based on a natural substrate based, for example, on mica coated with at least one layer of metal oxides.

The nacres according to the present disclosure may be characterized by a mean volume size of less than 15 microns, as measured with a laser granulometer, for instance, a Mastersizer 2000® from Malvern or a BI90+® from Brookhaven Instrument Corporation. The mica/tin oxide/titanium oxide nacres may include, in at least one embodiment Timiron Silk Blue®, Timiron Silk Red®, Timiron Silk Green®, Timiron Silk Gold®, and Timiron Super Silk® sold by the company Merck, and the mica/iron oxide/titanium oxide nacres may include, in at least one embodiment Flamenco Satin Blue®, Flamenco Satin Red®, and Flamenco Satin Violet® sold by the company Engelhard.

The composition according to the present disclosure may also contain various additional fillers chosen from mineral and organic fillers. They may be of any form, for example, platelet-shaped, spherical, and oblong, or may have another crystallographic form (for example leaflet, cubic, hexagonal, orthorhombic, etc.). Examples of suitable additional fillers that may be used in the composition according to the present disclosure include bismuth oxychloride, talc, mica, titanium dioxide, kaolin, poly-α-alanine and polyethylene particles, lauroyllysine, starch, boron nitride, precipitated calcium carbonate, magnesium carbonate, magnesium hydrogen carbonate, barium sulfate, hydroxyapatite, glass or ceramic microcapsules, and metal soaps derived from organic carboxylic acids comprising from 8 to 22 carbon atoms, for instance, from 12 to 18 carbon atoms, for example, zinc stearate, magnesium stearate, lithium stearate, zinc laurate, and magnesium myristate.

In at least one embodiment, the composition according to the present disclosure may comprise bismuth oxychloride, such as the bismuth oxychloride particles sold under the names BIRON LF 2000 and PEARL GLO SF by the companies Merck and Engelhard, respectively.

The at least one nacre and/or filler may be present in the composition in an amount, depending on the desired effect, ranging from 0.1% to 15%, for example, from 0.1% to 15%, or from 0.5% to 5%.

The composition in accordance with the present disclosure may also comprise at least one agent chosen from dyes, pigments, fragrances, preserving agents, physical and chemical sunscreens, sequestrants, liposoluble or water-soluble active agents, moisturizers such as polyols, for example, glycerol, and pH regulators (e.g., acids and bases).

The at least one agent may be present in the composition in an amount, for each of them, ranging from 0.01% to 20% by weight relative to the total weight of the composition.

It is to be understood that a person skilled in the art will take care to select the at least one optional additional compound such that the advantageous properties intrinsically associated with the composition in accordance with the present disclosure are not, or are not substantially, adversely affected by the envisaged addition.

The cosmetic compositions described in the present disclosure may be chosen from sera, aqueous gels, emulsions, sticks, and sprays. As used herein, the term “emulsion” means compositions comprising at least one aqueous phase and at least one oily phase dispersed in each other, and also water-in-oil-in-water and oil-in-water-in-oil multiple emulsions.

Also disclosed herein is a cosmetic—i.e., non-therapeutic—process for treating keratin materials, for example, the skin, comprising applying to the keratin materials a composition according to the present disclosure. Further disclosed herein is a method for reducing the appearance of skin marks and/or dyschromias and/or for lightening and/or unifying the complexion of the skin comprising applying to the skin a composition according to the present disclosure.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

I. Preparation of the Compositions

The compositions of the examples were prepared according to the following procedure:

heating phase B to about 75° C. and incorporating polyammonium acryldimethyltauramide therein; stirring until a homogeneous gel is obtained;

heating phase A to about 75° C.;

preparing the emulsion by incorporating phase A into phase B;

at 40-45° C., incorporating phase C and continuing stirring until cooling is complete.

II. Examples of Cosmetic Skincare Compositions

### Example 1

### Cosmetic Composition

A—

Glyceryl stearate (and) PEG-100 stearate: 2.00 g

Dimyristyl tartrate (and) cetearyl alcohol

(and) C12-15 pareth-7 (and) PPG-25 laureth-25: 1.50 g

Cyclohexasiloxane: 10.00 g

Stearyl alcohol: 1.00 g

B—

Water: 80.25 g

Vitamin C, 0.50 g

Phenoxyethanol: 1.00 g

Pentasodium ethylenediaminetetramethylenephosphate: 0.05 g

Polyammonium acryldimethyltauramide: 0.40 g

Xanthan gum: 0.20 g

C—

GOD BALL PC-LS-19

(iron oxide and titanium oxide encapsulated in silica

(45.8% TiO₂-50% silica-4.2% iron oxide)): 3.00 g

This composition had a value ΔE=42.3 and an opacity value of 22.9%.

### Example 2

### Cosmetic Composition

A—

Glyceryl stearate (and) PEG-100 stearate: 2.00 g

Dimyristyl tartrate (and) cetearyl alcohol

(and) C12-15 pareth-7 (and) PPG-25 laureth-25: 1.50 g

Cyclohexasiloxane: 10.00 g

Stearyl alcohol: 1.00 g

B—

Water: 80.25 g

Vitamin C, 0.50 g

Phenoxyethanol: 1.00 g

Pentasodium ethylenediaminetetramethylenephosphate: 0.05 g

Polyammonium acryldimethyltauramide: 0.40 g

Xanthan gum: 0.20 g

C—

GOD BALL PC-LS-14

(iron oxide and titanium oxide encapsulated in silica

(44.7% TiO₂-50% silica-5.3% iron oxide)): 3.00 g

This composition had a value ΔE=42.5 and an opacity value of 22.8%.

### Example 3

### Cosmetic Composition

A—

Glyceryl stearate (and) PEG-100 stearate: 2.00 g

Dimyristyl tartrate (and) cetearyl alcohol

(and) C12-15 pareth-7 (and) PPG-25 laureth-25: 1.50 g

Cyclohexasiloxane: 10.00 g

Stearyl alcohol: 1.00 g

B—

Water: 70.25 g

Ellagic acid: 0.50 g

Phenoxyethanol: 1.00 g

Pentasodium ethylenediaminetetramethylenephosphate: 0.05 g

Polyammonium acryldimethyltauramide: 0.40 g

Xanthan gum: 0.20 g

C—

GOD BALL PC-LS-14

(iron oxide and titanium oxide encapsulated in silica

(44.7% TiO₂-50% silica-5.3% iron oxide)): 2.00 g

Hollow hemispherical silicone particles

(NLK 506—Takemoto Oil and Fats): 3.00 g

This composition had a value ΔE=35.3 and an opacity value of 29.8%.

### Example 4

### Cosmetic Composition

A—

Glyceryl stearate (and) PEG-100 stearate: 2.00 g

Dimyristyl tartrate (and) cetearyl alcohol

(and) C12-15 pareth-7 (and) PPG-25 laureth-25: 1.50 g

Cyclohexasiloxane: 10.00 g

Stearyl alcohol: 1.00 g

B—

Water: 69.25 g

Kojic acid: 0.50 g

Phenoxyethanol: 1.00 g

Pentasodium ethylenediaminetetramethylenephosphate: 0.05 g

Polyammonium acryldimethyltauramide: 0.40 g

Xanthan gum: 0.20 g

C—

GOD BALL PC-LS-14

(iron oxide and titanium oxide encapsulated in silica

(44.7% TiO₂-50% silica-5.3% iron oxide)): 2.00 g

Hollow hemispherical silicone particles

(NLK 506-Takemoto Oil and Fats): 2.00 g

Mica/titanium oxide/tin oxide nacre

(Timiron Silk Blue): 1.00 g

This composition had a value ΔE=42 and an opacity value of 23.2%.

III. Demonstration of the Non-Covering Aspect of the Compositions According to the Invention

The compositions as described above were characterized by a color difference (ΔE) of greater than 30.

To determine this value, the composition was spread onto a transparent film (Hp Color laser jet transparency, Hp Invent; CP2936A) using an automatic applicator from Braive Instruments (wet thickness of 50 μm). The spreadings were then placed in a thermostatically maintained and ventilated oven for 24 hours at 37° C.

Once dried, these films were placed for evaluation on a contrast card (Prufkarte type 24/5-250 cm² sold by the company Erichsen). The color difference ΔE between the black part and the white part of the contrast card (measured through the film studied) was then measured with a Minolta CR-400 colorimeter. The color difference was obtained by means of Hunter's color difference formula in the L, a, b calorimetric space: ΔE=[(ΔL)²+(Δa)²+(Δb)²]^{0.5}. The values obtained were a mean of 5 measurements.

IV. Protocol for Measuring the Opacity of the Compositions According to the Invention

The compositions as described above are characterized by an opacity of less than 35%.

To determine this value, the composition was spread onto a transparent film (Hp Color laser jet transparency, Hp Invent; CP2936A) using an automatic applicator from Braive Instruments (wet thickness of 50 Δm). The spreadings were then placed in a thermostatically maintained and ventilated oven for 24 hours at 37° C.

Once dried, these films were placed for evaluation on a contrast card (Prufkarte type 24/5-250 cm² sold by the company Erichsen). The opacity was then measured using a Minolta CR-400 colorimeter from the values Y of the black and white parts obtained in the tristimulus system (X, Y, Z). The opacity value was obtained from the following equation:
(Y black area/Y white area)*100=% opacity

If the film is totally transparent, the opacity is equal to 0.

Sensory Evaluation:

The lightening and unifying effects and also the cosmetic aspects of the compositions according to the present disclosure (Examples 1 to 4) were evaluated on a panel of 6 individuals exhibiting dyschromias (pigmentation marks, depigmentation, freckles, couperose, and/or shadows under the eyes). For these persons, lightening, unifying effects, and smoothness of the grain of the skin were observed after application of the compositions according to the present disclosure, and blackheads are covered. The complexion was less blurred, and the color contrasts such as brown marks and undereye shadows were attenuated.

What is claimed is:

1. A cosmetic composition for treating and/or caring for the skin, comprising, in a physiologically acceptable medium:
(i) at least one first colored particle comprising at least one inorganic pigment arranged inside a matrix, and
(ii) at least one bleaching cosmetic active agent for the skin,
wherein the composition has an opacity of less than or equal to 35%.

2. A cosmetic composition for treating and/or caring for the skin, comprising, in a physiologically acceptable medium,
(i) an aqueous continuous phase,
(ii) at least one cosmetic active agent for the skin, and
(iii) at least one first colored particle comprising at least one inorganic pigment arranged inside a matrix,
wherein the composition has an opacity of less than or equal to 35%.

3. A cosmetic composition for treating and/or caring for the skin, comprising, in a physiologically acceptable medium,
(i) at least one cosmetic active agent for the skin, and
(ii) at least one first colored particle comprising at least two inorganic pigments of different colors arranged inside a matrix,
wherein the composition has an opacity of less than or equal to 35%.

4. The cosmetic composition according to claim 2, wherein the at least one cosmetic active agent is a bleaching cosmetic active agent.

5. The cosmetic composition according to claim 1, wherein the at least one bleaching cosmetic active agent is chosen from depigmenting agents, anti-pigmenting agents, pro-pigmenting agents, and mixtures thereof.

6. The cosmetic composition according to claim 1, wherein the bleaching cosmetic active agent is present in an amount ranging from 0.001% to 10% by weight relative to the total weight of the composition.

7. The cosmetic composition according to claim 6, wherein the bleaching cosmetic active agent is present in an amount ranging from 0.01% to 5% by weight relative to the total weight of the composition.

8. The cosmetic composition according to claim 1, further comprising an aqueous continuous phase.

9. The cosmetic composition according to claim 1, wherein the composition has an opacity ranging from 5% to 35%.

10. The cosmetic composition according to claim 9, wherein the composition has an opacity ranging from 10% to 30%.

11. The cosmetic composition according to claim 1, wherein the at least one first colored particle comprises at least two inorganic pigments of different colors arranged inside a matrix.

12. The cosmetic composition according to claim 1, wherein the at least one first colored particle comprises at least three inorganic pigments of different colors arranged inside a matrix.

13. The cosmetic composition according to claim 1, further comprising at least one additional second colored particle in an amount of less than or equal to 0.2% by weight relative to the total weight of the cosmetic composition.

14. The cosmetic composition according to claim 13, wherein the at least one additional second colored particle is chosen from organic pigments, inorganic pigments, and mixtures thereof.

15. The cosmetic composition according to claim 1, wherein the at least one inorganic pigment is chosen from red, yellow, blue, green, violet, brown, and black metal oxide pigments.

16. The cosmetic composition according to claim 1, wherein the at least one inorganic pigment is chosen from iron oxide, ultramarine, Prussian blue, ferric blue, manganese violet, chromium oxide, and mixed iron and titanium oxides.

17. The cosmetic composition according to claim 1, wherein the at least one inorganic pigment in the matrix of the at least one first colored particle is present in an amount ranging from 3% to 60% by weight relative to the total weight of the at least one first colored particle.

18. The cosmetic composition according to claim 17, wherein the at least one first colored particle is present in an amount ranging from 0.1% to 10% by weight relative to the total weight of the cosmetic composition.

19. The cosmetic composition according to claim 18, wherein the at least one first colored particle is present in an amount ranging from 0.5% to 5% by weight relative to the total weight of the cosmetic composition.

20. The cosmetic composition according to claim 1, further comprising at least one cosmetic active agent chosen from antioxidants, sunscreens, vitamins, moisturizing active agents, self-tanning compounds, anti-wrinkle active agents, hydrophilic or lipophilic active agents, anti-pollution agents, free-radical scavengers, dermo-relaxing active agents, calmatives, agents for stimulating the synthesis of dermal or epidermal macromolecules and/or for preventing their degradation, anti-glycation agents, anti-irritant agents, desquamating agents, NO-synthase inhibitors, agents for stimulating fibroblast or keratinocyte proliferation and/or keratinocyte differentiation, agents acting on the capillary circulation, agents acting on the energy metabolism of cells, cicatrizing agents, and mixtures thereof.

21. The cosmetic composition according to claim 1, further comprising at least one anti-aging cosmetic active agent chosen from compounds that increase collagen synthesis, compounds that inhibit collagen degradation, dermo-decontracting agents, protein glycation inhibitors, agents for increasing keratinocyte proliferation, agents for increasing keratinocyte differentiation, and mixtures thereof.

22. The cosmetic composition according to claim 1, wherein the at least one cosmetic active agent is present in an amount ranging from 0.001% to 10% by weight relative to the total weight of the composition.

23. The cosmetic composition according to claim 22, wherein the at least one cosmetic active agent is present in an amount ranging from 0.01% to 5% by weight relative to the total weight of the composition.

24. The cosmetic composition according to claim 1, further comprising at least one matting filler and/or nacre of small size.

25. The cosmetic composition according to claim 24, wherein the at least one matting filler and/or nacre is present in the composition in an amount ranging from 0.1% to 15% by weight relative to the total weight of the composition.

26. The cosmetic composition according to claim 1, further comprising at least one cosmetic additive chosen from dyes, hydrophilic or lipophilic gelling agents and/or thickeners, fragrances, preserving agents, neutralizers, emollients, sequestrants, film-forming agents, surfactants, and mixtures thereof.

27. The cosmetic composition according to claim 1, wherein the composition is in a form chosen from sera, aqueous gels, emulsions, sticks, and sprays.

28. The cosmetic composition according to claim 1, wherein the composition is in the form of an oil-in-water emulsion.

29. A cosmetic process for treating keratin materials comprising applying to the keratin materials a cosmetic composition comprising, in a physiologically acceptable medium:
(i) at least one first colored particle comprising at least one inorganic pigment arranged inside a matrix, and
(ii) at least one bleaching cosmetic active agent for the skin,
wherein the composition has an opacity of less than or equal to 35%.

30. The cosmetic process according to claim 29, wherein the keratin material is skin.

31. A cosmetic process for treating keratin materials comprising applying to the keratin materials a cosmetic composition comprising, in a physiologically acceptable medium:
(i) an aqueous continuous phase,
(ii) at least one cosmetic active agent for the skin, and
(iii) at least one first colored particle comprising at least one inorganic pigment arranged inside a matrix,
wherein the composition has an opacity of less than or equal to 35%.

32. A cosmetic process for treating keratin materials comprising applying to the keratin materials a cosmetic composition comprising, in a physiologically acceptable medium:
(i) at least one cosmetic active agent for the skin, and
(ii) at least one first colored particle comprising at least two inorganic pigments of different colors arranged inside a matrix,
wherein the composition has an opacity of less than or equal to 35%.

33. A method for reducing the appearance of skin marks and/or dyschromias comprising applying to the skin a cosmetic composition comprising, in a physiologically acceptable medium:
(i) at least one first colored particle comprising at least one inorganic pigment arranged inside a matrix, and
(ii) at least one bleaching cosmetic active agent for the skin,
wherein the composition has an opacity of less than or equal to 35%.

34. A method for reducing the appearance of skin marks and/or dyschromias comprising applying to the skin a cosmetic composition comprising, in a physiologically acceptable medium:
(i) an aqueous continuous phase,
(ii) at least one cosmetic active agent for the skin, and
(iii) at least one first colored particle comprising at least one inorganic pigment arranged inside a matrix,
wherein the composition has an opacity of less than or equal to 35%.

35. A method for reducing the appearance of skin marks and/or dyschromias comprising applying to the skin a cosmetic composition comprising, in a physiologically acceptable medium:
(i) at least one cosmetic active agent for the skin, and
(ii) at least one first colored particle comprising at least two inorganic pigments of different colors arranged inside a matrix,
wherein the composition has an opacity of less than or equal to 35%.

36. A method for lightening and/or unifying the complexion of the skin comprising applying to the skin a cosmetic composition comprising, in a physiologically acceptable medium:
(i) at least one first colored particle comprising at least one inorganic pigment arranged inside a matrix, and
(ii) at least one bleaching cosmetic active agent for the skin,
wherein the composition has an opacity of less than or equal to 35%.

37. A method for lightening and/or unifying the complexion of the skin comprising applying to the skin a cosmetic composition comprising, in a physiologically acceptable medium:
(i) an aqueous continuous phase,
(ii) at least one cosmetic active agent for the skin, and
(iii) at least one first colored particle comprising at least one inorganic pigment arranged inside a matrix,
wherein the composition has an opacity of less than or equal to 35%.

38. A method for lightening and/or unifying the complexion of the skin comprising applying to the skin a cosmetic composition comprising, in a physiologically acceptable medium:
(i) at least one cosmetic active agent for the skin, and
(ii) at least one first colored particle comprising at least two inorganic pigments of different colors arranged inside a matrix,
wherein the composition has an opacity of less than or equal to 35%.
